(19) United States
(12) Patent Application Publication
(10) Pub. No.: US 2021/0312579 A1
(43) Pub. Date: Oct. 7, 2021

(54) METHOD AND APPARATUS FOR ARRANGING AN EMERGENCY RESCUE STATION, SERVER, AND STORAGE MEDIUM

(71) Applicant: SOUTH UNIVERSITY OF SCIENCE AND TECHNOLOGY OF CHINA, Shenzhen (CN)

(72) Inventors: Xuan Song, Shenzhen (CN); Haoran Zhang, Shenzhen (CN); Ning Xu, Shenzhen (CN); Liqiao Huang, Shenzhen (CN); Zipei Fan, Shenzhen (CN); Ryosuke Shibasaki, Shenzhen (CN)

(21) Appl. No.: 17/224,631

(22) Filed: Apr. 7, 2021

(30) Foreign Application Priority Data
Apr. 7, 2020 (CN) 202010263747.1

(51) Int. Cl. G06Q 50/26; G16H 40/20; G06F 16/29; G06Q 10/04; G01C 21/34

(52) U.S. Cl. CPC G06Q 50/26; G16H 40/20; G01C 21/3446; G06Q 10/047; G01C 21/3407; G06F 16/29

(57) ABSTRACT

A method for arranging an emergency rescue station includes obtaining coordinates of at least one rescue center, coordinates of at least one hospital, and coordinates of at least one object to be rescued within a preset range; determining the number of remaining beds in the at least one hospital, and determining a rescue number corresponding to the at least one object to be rescued; determining whether the rescue number is less than or equal to the number of remaining beds; and in response to the rescue number being less than or equal to the number of remaining beds, determining coordinates of a newly added rescue center based on the coordinates of the at least one rescue center, the coordinates of the at least one hospital, and the coordinates of the at least one object to be rescued.

### CROSS-REFERENCE TO RELATED APPLICATION

This application is a U.S. application which claims the benefit and priority to Chinese patent application No. 202010263747.1 filed on Apr. 7, 2020 with China National Intellectual Property Administration, the entire contents of which are incorporated herein by reference.

### TECHNICAL FIELD

Embodiments of the present disclosure relate to big data technology, and more particularly relate to a method and apparatus for arranging an emergency rescue station, a server, and a storage medium.

### BACKGROUND

Disaster is a general term for things that can have a devastating impact on humans and the environment on which humans depend. Disaster does not indicate the degree, usually a local one that can expand and develop, and further evolve into a catastrophe. For example, widespread spread and prevalence of an infectious disease, fire, flood, and tsunami, or all natural events and social events that cause harm to natural ecological environment, material and spiritual civilization construction of human society, especially people's lives and property. Disasters are generally sudden, so doing a good job in the arrangement of emergency rescue stations in response to sudden disasters is of a particular importance.

Currently, layout and construction of rescue stations is typically determined based on a planner's experience and the density of distribution of objects to be rescued. However, such an arrangement method is prone to technical problems that rescue workers cannot be reasonably allocated and the rescue time is increased.

### SUMMARY

The present disclosure provides a method and apparatus for arranging an emergency rescue station, a server, and a storage medium, so as to achieve better allocation of rescue units and shorten rescue time to the greatest extent.

In a first aspect, embodiments of the present disclosure provide a method of arranging an emergency rescue station, the method comprising the following operations:

obtaining coordinates of at least one rescue center, coordinates of at least one hospital, and coordinates of at least one object to be rescued within a preset range;

determining the number of remaining beds in a hospital corresponding to the coordinates of the at least one hospital, and determining a rescue number corresponding to the at least one object to be rescued based on the coordinates of the at least one object to be rescued;

determining whether the rescue number is less than or equal to the number of remaining beds; and

in response to the rescue number being less than or equal to the number of remaining beds, determining coordinates of a newly added rescue center based on the coordinates of the at least one rescue center, the coordinates of the at least one hospital and the coordinates of the at least one object to be rescued.

Furthermore, the operation of obtaining the coordinates of the at least one rescue center, the coordinates of the at least one hospital, and the coordinates of the at least one object to be rescued within the preset range may include the following operations:

obtaining the coordinates of the at least one rescue center and the coordinates of the at least one hospital within the preset range according to a navigation positioning service or a preset data platform;

obtaining social networking information based on a preset social media platform and extracting social networking contents of the social networking information, the social networking contents comprising at least one keyword;

determining whether a target object corresponding to the social networking information is an object to be rescued according to the at least one keyword; and

in response to determining that the target object is an object to be rescued, determining the coordinates of the at least one object to be rescued according to the social networking contents.

Furthermore, the operation of determining the number of remaining beds in the hospital corresponding to the coordinates of the at least one hospital, and determining the rescue number corresponding to the at least one object to be rescued based on the coordinates of the at least one object to be rescued may include the following operations:

determining the number of remaining beds in the hospital corresponding to the coordinates of each of the at least one hospital through a preset medical information system and the coordinates of the at least one hospital; and

determining the rescue number of the at least one object to be rescued according to the coordinates of the at least one object to be rescued.

Furthermore, the operation of in response to the rescue number being less than or equal to the number of remaining beds, determining the coordinates of the newly added rescue center based on the coordinates of the at least one rescue center, the coordinates of the at least one hospital, and the coordinates of the at least one object to be rescued may include the following:

in response to the rescue number being less than or equal to the number of remaining beds, determining at least one driving route from the coordinates of the at least one object to be rescued to the coordinates of the at least one hospital based on the coordinates of the at least one object to be rescued and the coordinates of the at least one hospital;

determining a shortest driving route from the coordinates of the at least one object to be rescued to the coordinates of the at least one hospital based on the at least one driving route; and

determining the coordinates of the newly added rescue center using an ABLD (Accelerated Bi-level Decomposition) algorithm based on the shortest driving route and the coordinates of the at least one rescue center.

Furthermore, the method may further include the following subsequent to the operation of determining the coordinates of the newly added rescue center using the ABLD algorithm based on the shortest driving route and the coordinates of the at least one rescue center:

determining whether the coordinates of the newly added rescue center match the coordinates of the at least one rescue center based on a matching threshold value, and generating a matching degree; and

in response to the matching degree being greater than or equal to the matching threshold value, using the coordinates of the rescue center corresponding to the matching degree as the coordinates of the newly added rescue center.

Furthermore, the method may further include the following operations subsequent to the operation of determining whether the rescue number is less than or equal to the number of remaining beds:

in response to the rescue number being greater than the number of remaining beds, determining coordinates of a first nearest hospital based on the coordinates of the at least one object to be rescued and the coordinates of the at least one hospital;

determining a differential value between the rescue number and the number of remaining beds based on the coordinates of the first nearest hospital;

determining whether the differential value is less than the number of remaining beds;

in response to the differential value being less than the number of remaining beds, determining coordinates of a second nearest hospital based on the coordinates of the at least one object to be rescued and the coordinates of the at least one hospital; and

determining the coordinates of the newly added rescue center using an ABLD algorithm based on the coordinates of the first nearest hospital, the coordinates of the second nearest hospital, and the coordinates of the at least one object to be rescued.

In a second aspect, embodiments of the present disclosure further provide an apparatus for arranging an emergency rescue station, and the apparatus includes a coordinate acquisition module, a number determination module, a bed determination module, and a coordinate determination module.

The coordinate acquisition module is configured for obtaining coordinates of at least one rescue center, coordinates of at least one hospital, and coordinates of at least one object to be rescued within a preset range.

The number determination module is configured for determining a number of remaining beds in a hospital corresponding to the coordinates of the at least one hospital, and determining a rescue number corresponding to the at least one object to be rescued based on the coordinates of the at least one object to be rescued.

The bed determination module is configured for determining whether the rescue number is less than or equal to the number of remaining beds.

The coordinate determination module is configured for determining coordinates of a newly added rescue center based on the coordinates of the at least one rescue center, the coordinates of the at least one hospital, and the coordinates of the at least one object to be rescued in response to the rescue number being less than or equal to the number of remaining beds.

Furthermore, the coordinate acquisition module includes a coordinate acquisition unit.

The coordinate acquisition unit is configured for: obtaining the coordinates of the at least one rescue center and the coordinates of the at least one hospital within the preset range according to a navigation positioning service or a preset data platform; obtaining social networking information based on a preset social media platform and extracting social networking contents of the social networking information, the social networking contents comprising at least one keyword; determining whether a target object corresponding to the social networking information is an object to be rescued according to the at least one keyword; and determining the coordinates of the at least one object to be rescued according to the social networking contents in response to determining that the target object is an object to be rescued.

In a third aspect, embodiments of this disclosure further provide a server including one or more processors and a memory.

The memory is used for storing one or more programs.

When the one or more programs are executed by the one or more processors, the one or more processors is caused to perform the method of arranging an emergency rescue station according to any one of the embodiments described above.

In a fourth aspect, embodiments of this disclosure further provide a computer-readable storage medium configured to store one or more computer programs. When executed by a processor, the one or more programs perform the method of arranging an emergency rescue station according to any one of the above-mentioned embodiments.

In the present disclosure, the coordinates of the at least one object to be rescued, the coordinates of the at least one rescue center and the coordinates of the at least one hospital are obtained, so as to determine the rescue station to be newly added based on the distance from the coordinates of the at least one object to be rescued to the coordinates of the at least one hospital as well as the number of remaining beds. Thus, technical problems are solved that the arrangement of a preset newly added rescue station cannot be adjusted adaptively with respect to the coordinates of the at least one rescue center of the rescue station, the coordinates of the at least one hospital and a current position of the object to be rescued in the related art, and that rescue workers cannot be reasonably allocated and the rescue time is increased, thus achieving better allocation of the rescue units and shortening the rescue time to the largest extent.

### BRIEF DESCRIPTION OF DRAWINGS

FIG. 1 is a flowchart illustrating a method of arranging an emergency rescue station according to a first embodiment of the present disclosure.

FIG. 2 is a schematic diagram illustrating coordinates of objects to be rescued, coordinates of a rescue center, and coordinates of a hospital according to the first embodiment of the present disclosure.

FIG. 3 is a flowchart illustrating a method of arranging an emergency rescue station according to a second embodiment of the present disclosure.

FIG. 4 is a flowchart illustrating a method of arranging an emergency rescue station according to an alternative embodiment of the second embodiment of the present disclosure.

FIG. 5 is a block diagram illustrating an apparatus for arranging an emergency rescue station according to a third embodiment of the present disclosure.

FIG. 6 is a schematic diagram illustrating a server according to a fourth embodiment of the present disclosure.

### DETAILED DESCRIPTION

Hereinafter the present disclosure will be further described in detail in connection with the drawings and embodiments. It is to be understood that the specific embodiments set forth herein are merely intended to illustrate rather than limiting the present disclosure. Additionally, it is to be noted that for ease of description, merely part, not all, of the structures related to the present disclosure are illustrated in the drawings.

Before discussing exemplary embodiments in more detail, it is to be mentioned that some exemplary embodiments are described as processing or methods depicted as flowcharts. Although the flowchart describes the steps as sequential processing, many of them may be implemented concurrently, concomitantly or simultaneously. In addition, an order of the steps may be rearranged. The processing may be terminated when an operation is completed, but may also have additional steps not included in the drawings. The processing may correspond to methods, functions, procedures, subroutines, subprograms and the like.

In addition, terms “first”, “second” and the like may be used herein to describe various directions, actions, steps or elements, but these directions, actions, steps or elements are not limited by these terms. These terms are only used to distinguish a first direction, action, step or element from another direction, action, step or element. For example, without departing from the scope of the present application, coordinates of a first rescue center may be referred to as coordinates of a second rescue center, and similarly, coordinates of the second rescue center may be referred to as coordinates of the first rescue center. Both coordinates of the first rescue center and coordinates of the second rescue center are coordinates of a rescue center, but they do not refer to the coordinates of the same rescue center. The terms “first,” “second,” and the like cannot be understood as indicating or implying relative importance or implicitly illustrating a number of indicated technical features. Therefore features defined as “first” and “second” may explicitly or implicitly include one or more the features. In the description of the present disclosure, “multiple” means at least two, for example, two, three, or the like, unless otherwise expressly and specifically defined.

### First Embodiment

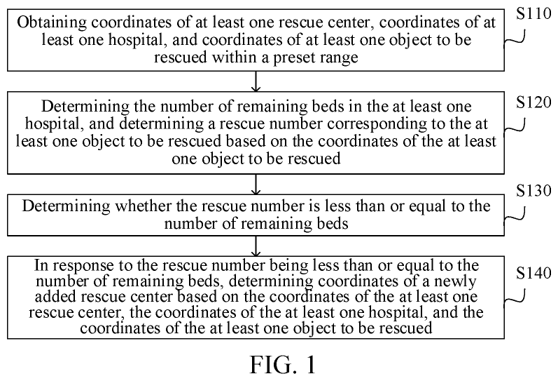

FIG. 1 is a flowchart illustrating a method of arranging an emergency rescue station according to a first embodiment of the present disclosure. This embodiment may be applied to how to build a more suitable rescue station according to a situation of a person to be rescued and a distribution of a hospital, and the method may be performed by a processor. As illustrated in FIG. 1, the method of arranging an emergency rescue station of this embodiment specifically includes the following operations.

In step S110, coordinates of at least one rescue center, coordinates of at least one hospital and coordinates of at least one object to be rescued within a preset range are obtained.

Specifically, a preset range here may be adjusted according to an accuracy required for layout optimization. The higher the required accuracy, the smaller the preset range, which is equivalent to dividing a large region into more small regions and arranging an emergency rescue station for each small region, and no further limitation is made here. The preset range of this embodiment may be configured or divided in a preset map. In this embodiment, a rescue station may be served as a transfer station, that is, objects to be rescued are concentrated in a rescue center, and then sent to a corresponding hospital from the rescue center uniformly, thus reducing the total time consumption for each object to be rescued to go to the corresponding hospital alone. Rescue center coordinates in this embodiment refer to geographic position coordinates of each rescue station used to receive the objects to be rescued within the preset range determined by the processor through a global position system (GPS) positioning navigation function and a preset algorithm (such as a particle swarm optimization (PSO) algorithm, a bi-level decomposition (BLD) algorithm, an accelerated bi-level decomposition (ABLD) algorithm, and the like). Hospital coordinates in this embodiment refer to geographical position coordinates of each hospital within the preset range determined by the processor through the GPS positioning navigation function and a preset algorithm (such as the PSO algorithm, the BLD algorithm, the ABLD algorithm, and the like). In this embodiment, coordinates of the objects to be rescued may refer to coordinates corresponding to current positions of persons to be rescued within the preset range (for example, a person who is located in a region where an epidemic or disaster occurs, a person suffering from a sudden disease, a person in a sick state, and the like). Coordinates here may refer to longitude and latitude, or three-dimensional coordinates established according to a unified coordinate system, or other coordinate representations used to distinguish different geographical positions, and a specific representation to be adopted is not limited here. In this embodiment, the coordinates of the objects to be rescued may be determined through social networking contents on a social platform (such as MicroBlog, Twitter, INS, WeChat, QQ, today's headlines, and the like), such as information for help and a current position of the object to be rescued published on the social platform, or a rescue news released by a certain official, where the rescue news includes a current position of a person to be rescued; and the social networking contents may also be screened on the social platform according to at least one keyword, so as to find out corresponding rescue information and determine the coordinates of the objects to be rescued according to a mobile terminal corresponding to the rescue information.

In this embodiment, the PSO algorithm is a population-based search process, in the PSO algorithm, each individual is called a particle and defined as a potential solution of an optimization problem in a D-dimensional search space, and a memory of its historical optimal position and optimal positions of all particles and a speed are retained. In each evolution generation, information of particles is combined to adjust a component of the speed on each dimension, and then the component is used to calculate a new particle position. Particles constantly change their states in a multi-dimensional search space until they reach equilibrium or optimal states or exceed computational limits. An only connection between different dimensions of a problem space is introduced through an objective function. The PSO algorithm includes, but is not limited to, a global particle swarm optimization (GPSO) algorithm, a local particle swarm optimization (LPSO) algorithm, a multi-swarm cooperative particle swarm optimization (MCPSO) algorithm, or a selective information particle swarm optimization (SIPSO) algorithm. A time relation of different PSO algorithms to determine a position of the rescue center is as follows: GPSO algorithm>MCPSO algorithm>LPSO algorithm>SIPSO algorithm. It takes the shortest time to determine the position of the rescue center by the SIPSO algorithm. However, GPSO exhibits a stronger convergence with respect to robustness in different test runs. In this embodiment, all regions that need to be optimized for emergency rescue station layout can be divided according to a preset range by a mixed-integer linear programming (MILP) model in advance to obtain a plurality of region blocks, and distances between the hospital, the object to be rescued and the region block needs to be determined, then a neuro-linguistic programming (NLP) model is established by the PSO algorithm or the BLD algorithm to solve an optimal value of rescue station layout optimization, such that a specific coordinate of the rescue station to be built on each area block in each area block is determined according to the NLP model, some rescue stations are built according to the specific coordinate, and then the processor obtains the rescue center coordinates of the built rescue station. Compared with the BLD algorithm, the ABLD algorithm is faster in solution speed. The ABLD algorithm is a kind of algorithm used to speed up a solution method. The ABLD algorithm is used to define a service range of hospitals and rescue stations, and a shortest distance is replaced by a median distance, such that difference between the MILP model and the NLP model is greatly reduced, and a predetermined optimal tolerance is reached many times.

In step S120, a number of remaining beds of a hospital corresponding to the coordinates of the at least one hospital is determined, and a rescue number corresponding to the at least one object to be rescued is determined based on the coordinates of the at least one object to be rescued.

Specifically, after the processor determines the hospital coordinates of each hospital within the preset range, the processor may further obtain hospital information of each hospital corresponding to the hospital coordinates through a pre-established cooperative relationship or an authorized big data platform, such as the number of remaining beds and distribution of the remaining beds (such as a first floor or a second floor, an emergency room or ordinary hospital bed, and the like), and the rescue number of the objects to be rescued is determined by a number of obtained coordinates of the objects to be rescued, that is, how many people need to be rescued corresponds to how many coordinates of the objects to be rescued.

In step S130, determination is made as to whether the rescue number is less than or equal to the number of remaining beds.

In S140, if the rescue number is less than or equal to the number of remaining beds, coordinates of a newly added rescue center is determined according to the coordinates of the at least one rescue center, the coordinates of the at least one hospital and the coordinates of the at least one object to be rescued.

Specifically, after determining the number of remaining beds in each hospital and the rescue number of the objects to be rescued, the processor further needs to compare the rescue number within the preset range with the number of remaining beds of each hospital to determine a hospital that can accept the rescue number of persons to be rescued, that is, the rescue number is less than or equal to the number of remaining beds. After determining a hospital that can be selected through the rescue number, the processor further needs to determine an optimal rescue center coordinates (that is, the coordinates of the newly added rescue center in this embodiment) corresponding to the shortest route of a hospital coordinate corresponding to the hospital that can be selected according to a hospital coordinate corresponding to the hospital that can be selected and each coordinate of the objects to be rescued based on the ABLD algorithm, where an object to be rescued is transported from the coordinate of the object to be rescued to the rescue center coordinates and then uniformly transported from the rescue center coordinates to the hospital that can be selected. After the processor solves the coordinates of the newly added rescue center through the ABLD algorithm and the NLP model, the coordinates of the newly added rescue center and the rescue center coordinates obtained before further need to be matched to determine whether the coordinates of the newly added rescue center is the rescue center coordinates obtained before. If so, the rescue center coordinates that is matched consistently may be served as the coordinates of the newly added rescue center, that is, there is no need to build a new emergency rescue station. If not, a new rescue station may be built according to the coordinates of the newly added rescue center, and the objects to be rescued are concentrated in the new rescue station and sent to the corresponding hospital along the shortest route.

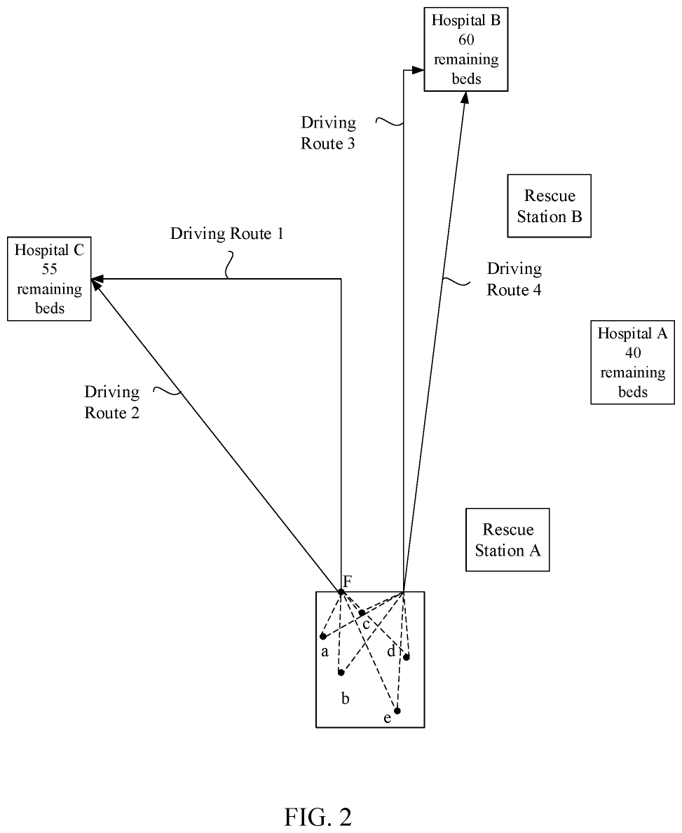

For example, FIG. 2 is a schematic diagram illustrating coordinates of objects to be rescued, the coordinates of the rescue center, and hospital coordinates according to the first embodiment of the present disclosure. As illustrated in FIG. 2, points of a, b, c, d and e are all coordinates of the objects to be rescued, each point may represent one object to be rescued or a set of a plurality of objects to be rescued that are close to each other (a distance threshold may be set here, that is, when a distance between the objects to be rescued is less than the distance threshold, the coordinates of the objects to be rescued may be processed according to a median value to unify into one coordinate, thus improving an overall operation efficiency), and here, for example, each point represents 10 persons. That is to say, the rescue number is 50. As can be seen from FIG. 2, only the number of remaining beds of hospital B and the number of remaining beds of hospital C are greater than the rescue number, and it is determined that the objects to be rescued of the points a, b, c, d and e may only be sent to the hospital B and the hospital C. There are two driving routes leading to the hospital B, namely, driving route 3 and driving route 4 respectively, and driving routes leading to the hospital C are driving route 1 and driving route 2 respectively. At this time, it is necessary to compare a driving distance of each driving route to determine the shortest driving route. As can be seen from FIG. 2, driving route 3 and driving route 4 are both longer, and the shortest route is driving route 2, that is to say, it takes the least time to take 50 persons to be rescued of the points a, b, c, d and e to the hospital C along driving route 2. At this time, it is also necessary to determine an overall driving distance from the points a, b, c, d and e to the hospital C respectively, that is, distances of segments of AF, BF, CF, DF and EF further need to be considered, and then through the ABLD algorithm and combined with a driving distance of driving route 2 and the distances of segments of AF, BF, CF, DF and EF, a coordinate of a point with a smallest total distance to points a, b, c, d and e and a hospital coordinate of the hospital C is finally determined, that is, the coordinates of the newly added rescue center of this embodiment (for example, point F in FIG. 2). As can be seen from FIG. 2, the coordinates of the newly added rescue center is not on rescue center coordinates corresponding to an built rescue station A and an built rescue station B, and therefore a new rescue station may be built according to the coordinates of the newly added rescue center when the emergency rescue station is arranged, so as to persons to be rescued of points a, b, c, d and e can reach the hospital C faster and the rescue time can be shortened.

Beneficial effects of the first embodiment of the present disclosure are described below. The coordinate of the object to be rescued, the rescue center coordinates and the hospital coordinate are acquired, and a position of the newly added rescue station is determined according to the distance from the coordinate of the object to be rescued to the hospital coordinate and the number of remaining beds. In this way, technical problems that a layout of a preset newly added rescue station cannot be adjusted adaptively for the rescue center coordinates of the rescue station, the hospital coordinate and a current position of the object to be rescued in the related art, rescue workers cannot be reasonably allocated and the rescue time is increased are solved, thus achieving a technical effect of better allocation of rescue units to shorten the rescue time to the greatest extent.

### Second Embodiment

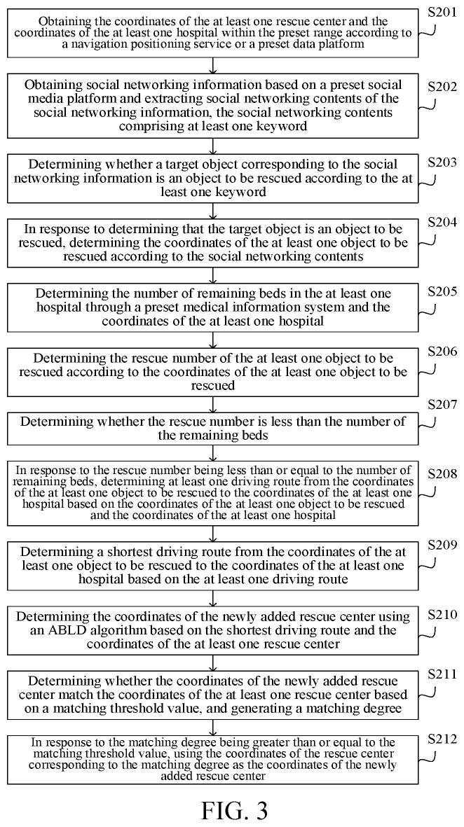

The second embodiment of the present disclosure is a further improvement on the basis of the first embodiment. FIG. 3 is a flowchart illustrating a method of arranging an emergency rescue station according to the second embodiment of the present disclosure. As illustrated in FIG. 3, the method of arranging an emergency rescue station of this embodiment includes the following operations.

In step S201, coordinates of at least one rescue center and coordinates of at least one hospital within a preset range are obtained according to a navigation positioning service or a preset data platform.

Specifically, a processor may obtain rescue center coordinates of all rescue stations and hospital coordinates of all hospitals within the preset range through a preset data platform (such as Baidu® maps, Google® maps, Amap® maps or other applications (APPs) or data platforms that can query corresponding coordinates) or the navigation and positioning service. In this embodiment, according to an epidemic situation or disaster situation in advance, all regions that need to be optimized for a layout of the emergency rescue station are divided according to the preset range to obtain a plurality of region blocks according to an MILP model; an NLP model for optimizing the layout of the rescue station to solve a better value is established through a PSO algorithm or a BLD algorithm, such that specific coordinates of a rescue station to be built on each region block in each region block are determined according to the NLP model, and the rescue station is built according to the specific coordinates. In general, the rescue stations will be built in a region where the objects to be rescued are relatively dense (may be determined by a density degree of obtained coordinates of the objects to be rescued), and then the processor can obtain the rescue center coordinates of the built rescue stations and the hospital coordinates of all hospitals within the preset range according to the navigation and positioning service or the preset data platform.

In step S202, social networking information is obtained based on a preset social media platform and social networking contents of the social networking information is extracted, where the social networking contents includes at least one keyword.

Specifically, the processor may preset a relevant keyword, and then parse acquired social networking contents on the preset social platform (such as MicroBlog®, Twitter®, Instagram®, WeChat®, QQ®, Jinri Toutiao®, and the like) to obtain at least one keyword of the social networking contents. In this embodiment, the processor may further acquire newly published social networking information on the social media platform according to a preset frequency.

In step S203, whether a target object corresponding to the social networking information is the object to be rescued is determined according to the at least one keyword.

Specifically, if the preset keywords are “rescue”, “ask for help”, “trapped”, “support”, “request” and the like, the processor may match the at least one keywords of the social networking information according to the words “rescue”, “ask for help”, “trapped”, “support”, and “request”. When the preset keywords match the at least one keywords of the social networking information, the target object corresponding to the social networking information may be considered as the object to be rescued. When the preset keywords and the at least one keywords of the social networking information are being matched, if the at least one keywords of social networking information are related to but different from the preset keywords, it may also be considered that the at least one keywords of the social networking information match the preset keywords. For example, the preset disaster keywords include fire, tsunami, flood and tornado, the at least one keywords of relevant social networking information include fire, thick smoke, smog and flood, then the fire, thick smoke and smoke may be considered to be related to the fire, and the preset keywords match the at least one keywords of the social networking information, and therefore the target object corresponding to the social networking information (that is, a person sending the social networking information or a person mentioned in the social networking information) may be taken as the object to be rescued.

In step S204, if the target object is the object to be rescued, the coordinates of the at least one object to be rescued are determined according to the social networking contents.

Specifically, if the target object corresponding to the social networking information is confirmed as the object to be rescued, the processor may determine a current position of the object to be rescued (that is, the coordinates of the objects to be rescued in this embodiment) according to the social networking contents of the social networking information. For example, the processor may acquire information for help (information including a geographic location, such as a certain street or a certain community) published by the object to be rescued on the social platform, and parse the information for help, thus obtaining the coordinates of the objects to be rescued; the processor determines the coordinates of the objects to be rescued according to the current position of the person to be rescued in a rescue news by acquiring the rescue news published by a certain official (the rescue news includes the current position of the person to be rescued); or the processor may further determine the coordinates of the objects to be rescued by positioning a current position of a mobile terminal of the object to be rescued corresponding to the social networking information.

In step S205, the number of remaining beds of the hospital corresponding to each of the hospital coordinates is determined based on a preset medical information system and the coordinates of the at least one hospital.

Specifically, after the processor determines hospital coordinates of each hospital and the coordinates of the objects to be rescued within the preset range, the processor may further acquire the number of remaining beds in each hospital corresponding to the hospital coordinates, and may further acquire distribution information of the remaining beds (such as a first floor or a second floor, an emergency room or an ordinary beds, and the like) through a preset medical information system or a pre-established cooperative relationship or an authorized big data platform.

In step S206, the rescue number of the object to be rescued is determined according to the coordinates of the objects to be rescued.

Specifically, the processor may determine the rescue number of the object to be rescued by acquired a number of coordinates of the objects to be rescued, that is, the rescue number is added by one every time one coordinate of the object to be rescued is acquired, and how many people need to be rescued corresponds to how many coordinates of the objects to be rescued. In this embodiment, when a distance of the coordinates of the objects to be rescued is less than a distance threshold, the object to be rescued may be regarded as a set, and a median value treatment is performed on the coordinates of the objects to be rescued, that is, a median value of the coordinates of the objects to be rescued is taken as a unified coordinate to calculate a distance between the set of the object to be rescued and the hospital, so as to improve an overall operation efficiency.

In step S207, determination is made as to whether the rescue number is less than or equal to the number of remaining beds.

In step S208, if the rescue number is less than or equal to the number of remaining beds, at least one driving route from the coordinates of the objects to be rescued to the hospital coordinates is determined according to the coordinates of the objects to be rescued and the hospital coordinates.

Specifically, after determining the number of remaining beds in each hospital and the rescue number of the objects to be rescued, the processor further needs to compare the rescue number within the preset range with the number of remaining beds of each hospital to determine a hospital that can accept the rescue number of persons to be rescued, that is, the rescue number is less than or equal to the number of remaining beds. After hospitals that can be selected are determined by the rescue number, the processor may further determine at least one driving route from the coordinates of the objects to be rescued to a hospital coordinate of a hospital according to each of the hospitals that can be selected.

In step S209, a shortest driving route from the coordinates of the objects to be rescued to the hospital coordinates is determined according to the at least one driving route.

Specifically, after determining at least one selectable driving route, the processor may further calculate driving distances of driving routes to different hospitals respectively according to the coordinates of the objects to be rescued, the hospital coordinate of the corresponding hospital and a traffic road map, so as to finally determine the shortest driving route with the least time consumption and the shortest driving distance among all driving routes.

In step S210, the coordinates of the newly added rescue center is determined using an ABLD algorithm according to the shortest driving route and the rescue center coordinates.

Specifically, the processor may further determine an optimal rescue center coordinates (that is, the coordinates of the newly added rescue center in this embodiment) corresponding to the shortest route of hospital coordinates corresponding to the hospitals that can be selected according to the hospital coordinates corresponding to the hospitals that can be selected and each coordinate of the objects to be rescued based on the ABLD algorithm, where an object to be rescued is transported from the coordinate of the object to be rescued to the rescue center coordinates and then uniformly transported from the rescue center coordinates to the hospitals that can be selected, that is, an optimal newly added rescue center coordinates (that is, every time the optimal newly added rescue center coordinates is calculated, rescue center coordinates of all rescue stations that have been built last time should be considered) is calculated in a superposition calculation mode through the ABLD algorithm and the rescue center coordinates of the built rescue stations.

In step S211, a determination is made as to whether the coordinates of the newly added rescue center match the rescue center coordinates according to a matching threshold value, and a matching degree is generated.

In step S212, if the matching degree is greater than or equal to the matching threshold value, the coordinates of the rescue center corresponding to the matching degree is taken as the coordinates of the newly added rescue center.

Specifically, the matching threshold may be adjusted according to an accuracy of the coordinates in this embodiment and is not limited herein. After the processor solves the coordinates of the newly added rescue center through the ABLD algorithm and an NLP model, the coordinates of the newly added rescue center and the rescue center coordinates obtained before further need to be matched to generate a matching degree (that is, similarity between the coordinates of the newly added rescue center and the rescue center coordinates), and whether the coordinates of the newly added rescue center is previous rescue center coordinates or very similar to the previous rescue center coordinates is determined. If so, the rescue center coordinates that is matched consistently may serve as the coordinates of the newly added rescue center, that is, there is no need to build a new emergency rescue station. If not, a new rescue station may be built according to the coordinates of the newly added rescue center, and the objects to be rescued are concentrated in the new rescue station and sent to a corresponding hospital along the shortest route.

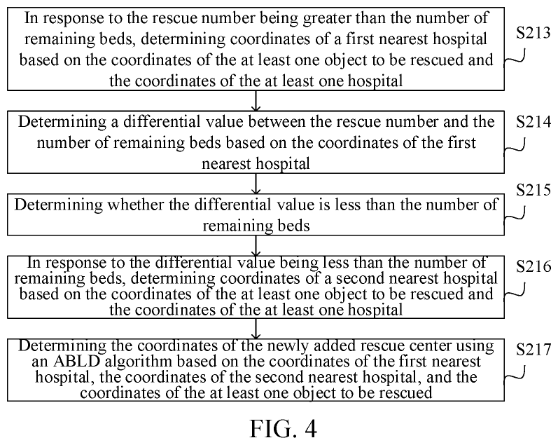

FIG. 4 is a flowchart illustrating a method of arranging an emergency rescue station according to an alternative embodiment of the second embodiment of the present disclosure. As shown in FIG. 4, the method may further include the following operations after step 207.

In step S213, if the rescue number is greater than the number of remaining beds, coordinates of a first nearest hospital are determined according to the coordinates of the objects to be rescued and the hospital coordinates.

Specifically, when the rescue number is greater than the number of remaining beds in each hospital, a hospital coordinate of a hospital closest to the coordinates of the objects to be rescued (that is, the coordinates of the first nearest hospital of this embodiment) may further be determined according to the coordinates of the objects to be rescued and the hospital coordinates.

In step S214, a differential value between the rescue number and the number of remaining beds is determined according to the coordinates of the first nearest hospital.

Specifically, after determining the coordinates of the first nearest hospital, the processor may further determine the differential value between the rescue number and the number of remaining beds of a hospital corresponding to the coordinates of the first nearest hospital, that is, how many beds are needed.

In step S215, a determination is made as to whether the differential value is less than the number of remaining beds.

In step S216, if the differential value is less than the number of remaining beds, coordinates of a second nearest hospital are determined according to the coordinates of the objects to be rescued and the hospital coordinates.

Specifically, after determining the differential value between the rescue number and the number of remaining beds of the hospital corresponding to the coordinates of the first nearest hospital by step S214, the processor may further compare the differential value with the number of remaining beds in other hospitals (that is, other than the hospital corresponding to the coordinates of the first nearest hospital); and when the differential value is less than the number of remaining beds in other hospitals, the processor may further determine a hospital coordinate corresponding to a hospital closest to the coordinates of the objects to be rescued (that is, the coordinates of the second nearest hospitals of this embodiment) according to hospital coordinates corresponding to hospitals screened and satisfying conditions and the coordinates of the objects to be rescued.

In step S217, the coordinates of the newly added rescue center is determined based on the ABLD algorithm according to the coordinates of the first nearest hospital, the coordinates of the second nearest hospital and the coordinates of the objects to be rescued.

Specifically, the processor may further determine a shortest driving route of two driving routes from the coordinates of the objects to be rescued to the coordinates of the first nearest hospital and from the coordinates of the first nearest hospital to the coordinates of the second nearest hospital according to the coordinates of the first nearest hospital, the coordinates of the second nearest hospital and each of the coordinates of objects to be rescued and based on the ABLD algorithm, and determine respective newly rescue center coordinates according to the shortest driving route. A step of determining the coordinates of the newly added rescue center is the same as step S210 to step S212, that is, the optimal newly added rescue center coordinates is calculated in the superposition calculation mode through the ABLD algorithm and the rescue center coordinates of the built rescue stations.

Beneficial effects of the second embodiment of the present disclosure are described below. The coordinates of the objects to be rescued, the rescue center coordinates and the hospital coordinates are obtained, and a point with a shortest total distance to the coordinates of the objects to be rescued and to the hospital coordinates is determined as the coordinates of the newly added rescue center of the newly added rescue station. In this way, technical problems are solved that a layout of a preset newly added rescue station cannot be adjusted adaptively for the rescue center coordinates of the rescue station, the hospital coordinate and a current position of the object to be rescued in the related art, rescue workers cannot be reasonably allocated and the rescue time is increased, thus achieving better allocation of rescue units and shortening the rescue time to the largest extent.

### Third Embodiment

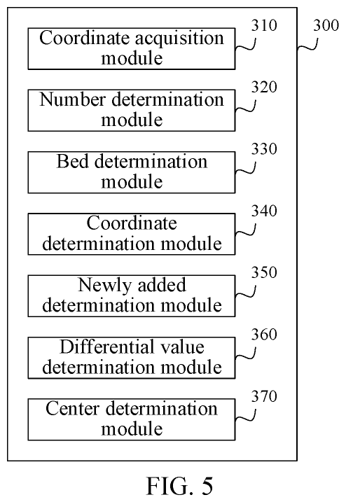

FIG. 5 is a schematic diagram illustrating an apparatus for arranging an emergency rescue station according to a third embodiment of the present disclosure. As illustrated in FIG. 5, the apparatus for arranging an emergency rescue station 300 of this embodiment includes a coordinate acquisition module 310, a number determination module 320, a bed determination module 330 and a coordinate determination module 340.

The coordinate acquisition module 310 is configured for obtaining coordinates of at least one rescue center, coordinates of at least one hospital and coordinates of at least one object to be rescued within a preset range.

The number determination module 320 is configured for determining the number of remaining beds of a hospital corresponding to the coordinates of the at least one hospital, and determining a rescue number corresponding to the object to be rescued according to the coordinates of the at least one object to be rescued.

The bed determination module 330 is configured for determining whether the rescue number is less than or equal to the number of remaining beds.

The coordinate determination module 340 is configured for determining, if the rescue number is less than or equal to the number of remaining beds, coordinates of a newly added rescue center according to the coordinates of the at least one rescue center, the coordinates of the at least one hospital and the coordinates of the at least one object to be rescued.

In this embodiment, the coordinate acquisition module 310 includes a coordinate acquisition unit.

The coordinate acquisition unit is configured for: obtaining the coordinates of the at least one rescue center and the coordinates of the at least one hospital within the preset range according to a navigation positioning service or a preset data platform; obtaining social networking information based on a preset social media platform and extracting social networking contents of the social networking information, the social networking contents including at least one keyword; determining whether a target object corresponding to the social networking information is an object to be rescued according to the at least one keyword; and if the target object is an object to be rescued, determining the coordinates of the at least one object to be rescued according to the social networking contents.

In this embodiment, the number determination module 320 includes a number determination unit.

The number determination unit is configured for determining the number of remaining beds of the hospital corresponding to the coordinates of each of the at least one hospital through a preset medical information system and the coordinates of the at least one hospital, and determining the rescue number of the object to be rescued according to the coordinates of the at least one object to be rescued.

In this embodiment, the coordinate determination module 340 includes a driving route determination unit.

The driving route determination unit is configured for determining, if the rescue number is less than or equal to the number of remaining beds, at least one driving route from the coordinates of the at least one object to be rescued to the coordinates of the at least one hospital according to the coordinates of the at least one object to be rescued and the coordinates of the at least one hospital; and determining a shortest driving route from the coordinates of the at least one object to be rescued to the coordinates of the at least one hospital according to the at least one driving route.

The coordinate determination unit is configured for determining the coordinates of the newly added rescue center based on an ABLD algorithm according to the shortest driving route and the rescue center coordinates.

In this embodiment, the apparatus for arranging an emergency rescue station 300 further includes a newly added determination module 350.

The newly added determination module 350 is configured for: determining whether the coordinates of the newly added rescue center match the coordinates of the at least one rescue center according to a matching threshold value, and generating a matching degree; and if the matching degree is greater than or equal to the matching threshold value, taking the coordinates of the rescue center corresponding to the matching degree as the coordinates of the newly added rescue center.

In this embodiment, the apparatus for arranging an emergency rescue station 300 further includes a differential value determination module 360 and a center determination module 370.

The differential value determination module 360 is configured for if the rescue number is greater than the number of remaining beds, determining coordinates of a first nearest hospital according to the coordinates of the at least one object to be rescued and the coordinates of the at least one hospital; and determining a differential value between the rescue number and the number of remaining beds according to the coordinates of the first nearest hospital.

The center determination module 370 is configured for determining whether the differential value is less than the number of remaining beds; if the differential value is less than the number of remaining beds, determining coordinates of a second nearest hospital according to the coordinates of the at least one object to be rescued and the coordinates of the at least one hospital; and determining the coordinates of the newly added rescue center based on the ABLD algorithm according to the coordinates of the first nearest hospital, the coordinates of the second nearest hospital and the coordinates of the at least one object to be rescued.

The emergency rescue station layout system provided by the embodiment of the present disclosure can execute the method of arranging an emergency rescue station provided by any one of embodiments of the present disclosure, and has corresponding functional modules provide corresponding beneficial effects of the method.

### Fourth Embodiment

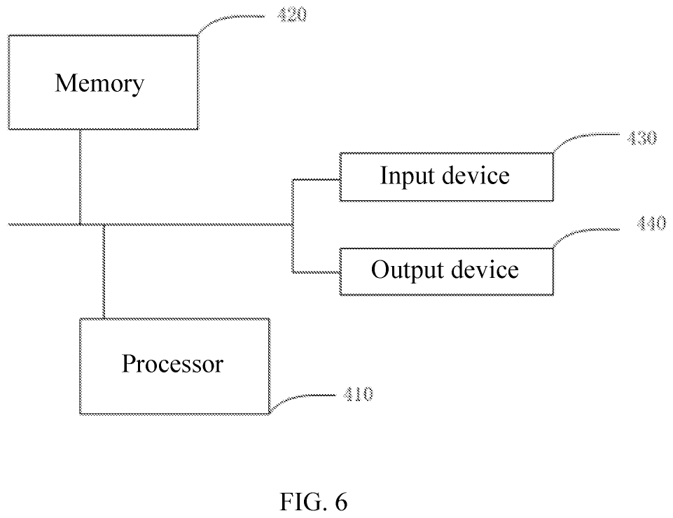

FIG. 6 is a schematic diagram illustrating a server according to a fourth embodiment of the present disclosure. As illustrated in FIG. 6, the server includes a processor 410, a memory 420, an input apparatus 430 and an output apparatus 440. A number of processors 410 in the server may be one or more, and one processor 410 is described an example in FIG. 6. The processor 410, the memory 420, the input apparatus 430, and the output apparatus 440 in the server may be connected by a bus or other ways, and connected by the bus is described as an example in FIG. 6.

The memory 420 serves as a computer-readable storage medium and may be used for storing software programs, computer executable programs and modules, such as program instructions/modules corresponding to the emergency rescue station layout system in the embodiment of the present disclosure (for example, a coordinate acquisition module, a number determination module, a bed determination module, a coordinate determination module, a newly added determination module, a differential value determination module and a center determination module in an apparatus for arranging an emergency rescue station). The processor 410 executes various functional applications and data processing of the server by running the software programs, instructions and modules stored in the memory 420, that is, implements the above-mentioned method of arranging an emergency rescue station, and the method of arranging an emergency rescue station is described below.

Coordinates of at least one rescue center, coordinates of at least one hospital and coordinates of at least one object to be rescued within a preset range are obtained.

The number of remaining beds in a hospital corresponding to the coordinates of the at least one hospital is determined, and the rescue number corresponding to the at least one object to be rescued is determined according to the coordinates of the at least one object to be rescued.

A determination is made as to whether the rescue number is less than or equal to the number of remaining beds.

If the rescue number is less than or equal to the number of remaining beds, coordinates of a newly added rescue center is determined according to the coordinates of the at least one rescue center, the coordinates of the at least one hospital and the coordinates of the at least one object to be rescued.

The memory 420 may mainly include a program storing region and a data storing region, where the program storing region may store an operating system, application programs required for at least one function; and the data storing region may store data created according to use of a terminal. In addition, the memory 420 may include a high-speed random access memory and may further include a nonvolatile memory, such as at least one magnetic storage apparatus, flash memory, or other nonvolatile solid-state memory. In some embodiments, the memory 420 may further include a memory remotely disposed relative to the processor 410, and the remote memory may be connected to the server through a network. Examples of the above-mentioned network include, but are not limited to, the Internet, an intranet, a local area network, a mobile communication network, and combinations thereof.

The input apparatus 430 may be used to receive inputted numeric or character information and to generate a key signal input related to user settings and function control of the server. The output apparatus 440 may include display devices such as a display screen.

### Fifth Embodiment

The fifth embodiment of the present disclosure further provides a storage medium including computer-executable instructions for executing a method of arranging an emergency rescue station when executed by a computer processor, the method including the following operations.

Coordinates of at least one rescue center, coordinates of at least one hospital and coordinates of at least one object to be rescued within a preset range are obtained.

The number of remaining beds in a hospital corresponding to the coordinates of the at least one hospital is determined, and the rescue number corresponding to the object to be rescued is determined according to the coordinates of the at least one object to be rescued.

A determination is made as to whether the rescue number is less than or equal to the number of remaining beds.

If the rescue number is less than or equal to the number of remaining beds, coordinates of a newly added rescue center is determined based on the coordinates of the at least one rescue center, the coordinates of the at least one hospital and the coordinates of the at least one object to be rescued.

Of course, for the storage medium including the computer-executable instructions provided by the embodiment of the present disclosure, the computer-executable instructions are not limited to the above-mentioned method operation, and may further execute related operations in the method of arranging an emergency rescue station provided by any embodiment of the present disclosure.

From the description of the embodiments described above, it will be apparent to those skilled in the art that the present disclosure may be implemented by software plus a necessary general-purpose hardware platform, or may of course be implemented by hardware. However, in many cases, the former is a preferred implementation manner. Based on this understanding, the substantial technical solution of the present disclosure, or the part contributing to the related art, may be embodied in the form of a software product. Computer software products may be stored in a computer-readable storage medium, such as a floppy disk of a computer, a read-only memory (ROM), a random access memory (RAM), a flash memory (FLASH), a hard disk, an optical disk or the like, and include multiple instructions for enabling a computer apparatus (which may be a personal computer, a server, a network apparatus or the like) to perform the method according to each embodiment of the present disclosure.

It is to be noted that in the above-mentioned embodiment of the apparatus for arranging an emergency rescue station, each unit and module included are only divided according to functional logic, but are not limited to the above division, as long as corresponding functions can be implemented. Moreover, the specific names of each functional unit are only for convenience of distinguishing each other and are not used to limit the scope of protection of the present disclosure.

It is to be noted that the foregoing merely depict some illustrative embodiments according to this disclosure and the technical principles used herein. It will be understood by those skilled in the art that the present disclosure will not be limited to the specific embodiments described herein. Those skilled in the art can make various apparent modifications, adaptations and substitutions without departing from the scope of this disclosure. Thus, while thus disclosure has been described in detail through the above-described embodiments, the present disclosure will not be limited to these embodiments and may further include other additional equivalent embodiments without departing from the concept of the present disclosure. The scope of the present disclosure is determined in and by the appended claims.

1. A method of arranging an emergency rescue station, comprising:
obtaining coordinates of at least one rescue center, coordinates of at least one hospital, and coordinates of at least one object to be rescued within a preset range;
determining a number of remaining beds in the at least one hospital, and determining a rescue number corresponding to the at least one object to be rescued based on the coordinates of the at least one object to be rescued;
determining whether the rescue number is less than or equal to the number of remaining beds; and
in response to the rescue number being less than or equal to the number of remaining beds, determining coordinates of a newly added rescue center based on the coordinates of the at least one rescue center, the coordinates of the at least one hospital, and the coordinates of the at least one object to be rescued.

2. The method of claim 1, wherein obtaining the coordinates of the at least one rescue center, the coordinates of the at least one hospital, and the coordinates of the at least one object to be rescued within the preset range comprises:
obtaining the coordinates of the at least one rescue center and the coordinates of the at least one hospital within the preset range according to a navigation positioning service or a preset data platform;
obtaining social networking information based on a preset social media platform and extracting social networking contents of the social networking information, the social networking contents comprising at least one keyword;
determining whether a target object corresponding to the social networking information is an object to be rescued according to the at least one keyword; and
in response to determining that the target object is an object to be rescued, determining the coordinates of the at least one object to be rescued according to the social networking contents.

3. The method of claim 1, wherein determining the number of remaining beds in the at least one hospital, and determining the rescue number corresponding to the at least one object to be rescued based on the coordinates of the at least one object to be rescued comprises:
determining the number of remaining beds in the at least one hospital through a preset medical information system and the coordinates of the at least one hospital; and
determining the rescue number of the at least one object to be rescued according to the coordinates of the at least one object to be rescued.

4. The method of claim 1, wherein in response to the rescue number being less than or equal to the number of remaining beds, determining the coordinates of the newly added rescue center based on the coordinates of the at least one rescue center, the coordinates of the at least one hospital, and the coordinates of the at least one object to be rescued comprises:
in response to the rescue number being less than or equal to the number of remaining beds, determining at least one driving route from the coordinates of the at least one object to be rescued to the coordinates of the at least one hospital based on the coordinates of the at least one object to be rescued and the coordinates of the at least one hospital;
determining a shortest driving route from the coordinates of the at least one object to be rescued to the coordinates of the at least one hospital based on the at least one driving route; and
determining the coordinates of the newly added rescue center using an ABLD (Accelerated Bi-level Decomposition) algorithm based on the shortest driving route and the coordinates of the at least one rescue center.

5. The method of claim 4, further comprising, subsequent to the operation of determining the coordinates of the newly added rescue center using the ABLD algorithm based on the shortest driving route and the coordinates of the at least one rescue center:
determining whether the coordinates of the newly added rescue center match the coordinates of the at least one rescue center based on a matching threshold value, and generating a matching degree; and
in response to the matching degree being greater than or equal to the matching threshold value, using the coordinates of the rescue center corresponding to the matching degree as the coordinates of the newly added rescue center.

6. The method of claim 1, further comprising, subsequent to the operation of determining whether the rescue number is less than or equal to the number of remaining beds:
in response to the rescue number being greater than the number of remaining beds, determining coordinates of a first nearest hospital based on the coordinates of the at least one object to be rescued and the coordinates of the at least one hospital;
determining a differential value between the rescue number and the number of remaining beds based on the coordinates of the first nearest hospital;
determining whether the differential value is less than the number of remaining beds;
in response to the differential value being less than the number of remaining beds, determining coordinates of a second nearest hospital based on the coordinates of the at least one object to be rescued and the coordinates of the at least one hospital; and
determining the coordinates of the newly added rescue center using an ABLD algorithm based on the coordinates of the first nearest hospital, the coordinates of the second nearest hospital, and the coordinates of the at least one object to be rescued.

7. An apparatus for arranging an emergency rescue station, comprising:
a coordinate acquisition module, configured for obtaining coordinates of at least one rescue center, coordinates of at least one hospital, and coordinates of at least one object to be rescued within a preset range;
a number determination module, configured for determining a number of remaining beds in the at least one hospital, and determining a rescue number corresponding to the at least one object to be rescued based on the coordinates of the at least one object to be rescued;
a bed determination module, configured for determining whether the rescue number is less than or equal to the number of remaining beds; and
a coordinate determination module, configured for determining coordinates of a newly added rescue center based on the coordinates of the at least one rescue center, the coordinates of the at least one hospital, and the coordinates of the at least one object to be rescued in response to the rescue number being less than or equal to the number of remaining beds.

8. The apparatus of claim 7, wherein the coordinate acquisition module comprises:
a coordinate acquisition unit, configured for: obtaining the coordinates of the at least one rescue center and the coordinates of the at least one hospital within the preset range according to a navigation positioning service or a preset data platform; obtaining social networking information based on a preset social media platform and extracting social networking contents of the social networking information, the social networking contents comprising at least one keyword; determining whether a target object corresponding to the social networking information is an object to be rescued according to the at least one keyword; and determining the coordinates of the at least one object to be rescued according to the social networking contents in response to determining that the target object is an object to be rescued.

9. The apparatus of claim 7, wherein the number determination module comprises a number determination unit configured for determining the number of remaining beds in the hospital corresponding to the coordinates of each of the at least one hospital through a preset medical information system and the coordinates of the at least one hospital, and determining the rescue number of the at least one object to be rescued according to the coordinates of the at least one object to be rescued.

10. The apparatus of claim 7, wherein the coordinate determination module comprises:
a driving route determination unit configured for: in response to the rescue number being less than or equal to the number of remaining beds, determining at least one driving route from the coordinates of the at least one object to be rescued to the coordinates of the at least one hospital based on the coordinates of the at least one object to be rescued and the coordinates of the at least one hospital; and determining a shortest driving route from the coordinates of the at least one object to be rescued to the coordinates of the at least one hospital based on the at least one driving route; and
a coordinate determination unit configured for determining the coordinates of the newly added rescue center using an ABLD (Accelerated Bi-level Decomposition) algorithm based on the shortest driving route and the coordinates of the at least one rescue center.

11. The apparatus of claim 10, further comprising a newly added determination module configured for:
determining whether the coordinates of the newly added rescue center match the coordinates of the at least one rescue center based on a matching threshold value, and generating a matching degree; and
in response to the matching degree being greater than or equal to the matching threshold value, using the coordinates of the rescue center corresponding to the matching degree as the coordinates of the newly added rescue center.

12. The apparatus of claim 7, further comprising:
a differential value determination module configured for determining coordinates of a first nearest hospital based on the coordinates of the at least one object to be rescued and the coordinates of the at least one hospital in response to the rescue number being greater than the number of remaining beds; and
a center determination module configured for: determining a differential value between the rescue number and the number of remaining beds based on the coordinates of the first nearest hospital; determining whether the differential value is less than the number of remaining beds; in response to the differential value being less than the number of remaining beds, determining coordinates of a second nearest hospital based on the coordinates of the at least one object to be rescued and the coordinates of the at least one hospital; and determining the coordinates of the newly added rescue center using an ABLD algorithm based on the coordinates of the first nearest hospital, the coordinates of the second nearest hospital, and the coordinates of the at least one object to be rescued.

13. A server, comprising:
one or more processors;
a memory configured for storing one or more programs, wherein the one or more programs when executed by the one or more processors causes the one or more processors to perform the method of claim 1.

14. The server of claim 13, wherein obtaining the coordinates of the at least one rescue center, the coordinates of the at least one hospital, and the coordinates of the at least one object to be rescued within the preset range comprises:
obtaining the coordinates of the at least one rescue center and the coordinates of the at least one hospital within the preset range according to a navigation positioning service or a preset data platform;
obtaining social networking information based on a preset social media platform and extracting social networking contents of the social networking information, the social networking contents comprising at least one keyword;
determining whether a target object corresponding to the social networking information is an object to be rescued according to the at least one keyword; and
in response to determining that the target object is an object to be rescued, determining the coordinates of the at least one object to be rescued according to the social networking contents.

15. The server of claim 13, wherein determining the number of remaining beds in the hospital corresponding to the coordinates of the at least one hospital, and determining the rescue number corresponding to the at least one object to be rescued based on the coordinates of the at least one object to be rescued comprises:
determining the number of remaining beds in the hospital corresponding to the coordinates of each of the at least one hospital through a preset medical information system and the coordinates of the at least one hospital; and
determining the rescue number of the at least one object to be rescued according to the coordinates of the at least one object to be rescued.

16. The server of claim 13, wherein in response to the rescue number being less than or equal to the number of remaining beds, determining the coordinates of the newly added rescue center based on the coordinates of the at least one rescue center, the coordinates of the at least one hospital, and the coordinates of the at least one object to be rescued comprises:
in response to the rescue number being less than or equal to the number of remaining beds, determining at least one driving route from the coordinates of the at least one object to be rescued to the coordinates of the at least one hospital based on the coordinates of the at least one object to be rescued and the coordinates of the at least one hospital;
determining a shortest driving route from the coordinates of the at least one object to be rescued to the coordinates of the at least one hospital based on the at least one driving route; and
determining the coordinates of the newly added rescue center using an ABLD (Accelerated Bi-level Decomposition) algorithm based on the shortest driving route and the coordinates of the at least one rescue center.

17. A computer-readable storage medium, storing one or more computer programs, which when executed by a processor causes processor to perform the method of claim 1.

18. The computer-readable storage medium of claim 17, wherein obtaining the coordinates of the at least one rescue center, the coordinates of the at least one hospital, and the coordinates of the at least one object to be rescued within the preset range comprises:
obtaining the coordinates of the at least one rescue center and the coordinates of the at least one hospital within the preset range according to a navigation positioning service or a preset data platform;
obtaining social networking information based on a preset social media platform and extracting social networking contents of the social networking information, the social networking contents comprising at least one keyword;
determining whether a target object corresponding to the social networking information is an object to be rescued according to the at least one keyword; and
in response to determining that the target object is an object to be rescued, determining the coordinates of the at least one object to be rescued according to the social networking contents.

19. The computer-readable storage medium of claim 17, wherein determining the number of remaining beds in the hospital corresponding to the coordinates of the at least one hospital, and determining the rescue number corresponding to the at least one object to be rescued based on the coordinates of the at least one object to be rescued comprises:
determining the number of remaining beds in the hospital corresponding to the coordinates of each of the at least one hospital through a preset medical information system and the coordinates of the at least one hospital; and
determining the rescue number of the at least one object to be rescued according to the coordinates of the at least one object to be rescued.

20. The computer-readable storage medium of claim 17, wherein in response to the rescue number being less than or equal to the number of remaining beds, determining the coordinates of the newly added rescue center based on the coordinates of the at least one rescue center, the coordinates of the at least one hospital, and the coordinates of the at least one object to be rescued comprises:
in response to the rescue number being less than or equal to the number of remaining beds, determining at least one driving route from the coordinates of the at least one object to be rescued to the coordinates of the at least one hospital based on the coordinates of the at least one object to be rescued and the coordinates of the at least one hospital;
determining a shortest driving route from the coordinates of the at least one object to be rescued to the coordinates of the at least one hospital based on the at least one driving route; and
determining the coordinates of the newly added rescue center using an ABLD (Accelerated Bi-level Decomposition) algorithm based on the shortest driving route and the coordinates of the at least one rescue center.
